(19) United States
(12) Patent Application Publication
(10) Pub. No.: US 2018/0021540 A1
(43) Pub. Date: Jan. 25, 2018

(54) VALVES AND TUBES INCLUDING VALVES

(71) Applicant: SMITHS MEDICAL INTERNATIONAL LIMITED, Ashford (GB)

(72) Inventors: Andrew Thomas Jeffrey, Hythe (GB); Christopher John Woosnam, Great Sutton (GB)

(73) Assignee: SMITHS MEDICAL INTERNATIONAL LIMITED, Ashford (GB)

(21) Appl. No.: 15/549,533

(22) PCT Filed: Jan. 19, 2016
(86) PCT No.: PCT/GB2016/000011

§ 371 Date: Aug. 8, 2017

(30) Foreign Application Priority Data
Feb. 17, 2015 (GB) 1502626.3

(51) Int. Cl. A61M 16/20; A61M 16/04

(52) U.S. Cl. CPC A61M 16/208; A61M 16/044

(57) ABSTRACT

A valve (20) for the sealing cuff (10) of a tracheal tube has a valve member (40) urged against a valve seal (36, 37) by an MRI-safe resilient member (42) of neoprene or nitrile. The valve includes a two-part actuator (70) by which the valve member is displaced. Before use, during storage, the valve member is in a closed position with the resilient member relatively relaxed. When the valve is opened for the first time by inserting a syringe or the like, the two-part actuator arrangement is moved forwards to unseat the valve member to allow flow. This causes inclined surfaces (88) and (92) on the two parts (72) and (73) of the actuator to rotate the forward part slightly. When the syringe is removed to allow the valve to close, the forward part of the actuator latches on a surface (34) of the housing (21) in an operational position where the valve is closed.

This invention relates to valves of the kind including an outer housing, a valve seat, a valve member displaceable along the housing into or out of sealing engagement with the valve seat, and a resilient member arranged to urge the valve member into engagement with the seat.

Various medico-surgical tubes, such as tracheal tubes, have an inflatable sealing cuff towards the patient end that can be inflated to seal the outside of the tube with a body cavity, such as the trachea. The cuff is inflated and deflated via a small bore lumen extending along the tube that connects towards its machine end with an inflation line terminated by an inflation indicator and a valve. The valve is arranged to remain closed, to block escape of air from the cuff during use but can be opened by inserting the nose of a syringe or a similar inflation device into the valve to displace a valve element away from a valve seat and allow air to be supplied to or from the valve to inflate or deflate the sealing cuff. It is important that the valve work reliably so that air does not leak out from the cuff and so that it can be opened readily to allow the cuff to be deflated, such as when the tube needs to be removed. Usually, these valves include a spring element in the form of a helical metal wire to urge the valve element to the seated, closed position. One problem with valves including such a spring element is that they may not be compatible with MRI scanners. Although it is possible to use a non-metallic spring element, such as of a resilient plastics material, these can be difficult to manufacture reliably. In particular, thermoplastics can be susceptible to shrinkage after removal from the mould or as a result of sterilisation or ageing after storage.

Valves are also used in other applications in addition to sealing cuffs of medico-surgical tubes.

It is an object of the present invention to provide an alternative valve and a medico-surgical tube including such a valve.

According to one aspect of the present invention there is provided a valve of the above-specified kind, characterised in that the valve includes an actuator arrangement extending from the valve member to an opening of the housing, and that the actuator arrangement includes a first part displaceable from an initial position where the valve is closed to a second position where the valve is also closed and is subsequently displaceable from the second position to a third position where the valve member is displaced against the action of the resilient member away from the seat to open the valve and allow flow through the housing, the first part of the actuator arrangement returning to the second position when released.

The actuator arrangement preferably includes a second part displaceable axially relative to the first part, the valve being opened by pushing the second part axially relative to the housing. The forward end of the second part and the rear end of the first part preferably have engaging inclined surfaces such that forward displacement of the second part can apply a rotational force to the first part. The second part may have at least one outwardly-projecting member arranged to engage in a longitudinal groove along a part of the housing such as to enable longitudinal displacement of the second part but to prevent rotational movement. The first part preferably has a plurality of outwardly-projecting members arranged to engage in respective longitudinal grooves along a part of the housing to prevent rotational movement when the valve is closed but to disengage the grooves when the actuator arrangement is engaged to open the valve and enable rotation of the first part to a position where the members engage on a latching formation when the actuator arrangement is released and thereby hold the first part in the second position. The resilient member is preferably of a non-metallic material. The resilient member may have a drum shape.

According to another aspect of the present invention there is provided valve including an outer housing, a valve seat, a valve member displaceable along the housing into or out of sealing engagement with the valve seat, a resilient member arranged to urge the valve member into engagement with the seat, characterised in that the outer housing has an internal latching formation, that the valve includes an actuator arrangement extending from the valve member to an opening of the housing such that the actuator arrangement can be displaced by extending a member into the opening of the housing, that the actuator arrangement includes a first part that is arranged to engage with the latching formation on the housing, that the first part of the actuator arrangement is displaceable from an initial position where the valve is closed to a second position where the valve is also closed and where the actuator arrangement engages with the latching formation to prevent subsequent displacement back to the initial position, that the first part of the actuator arrangement is displaceable from the second position away from the latching formation to a third position where the valve member is displaced against the action of the resilient member away from the seat to allow flow through the housing, and that the first part of the actuator arrangement is arranged to return to the second position in engagement with the latching formation when released.

According to a further aspect of the present invention there is provided a valve including an outer housing, a valve seat, a valve member displaceable along the housing into or out of sealing engagement with the valve seat, a resilient member arranged to urge the valve member into engagement with the seat, characterised in that the housing includes an internal latching formation, that the valve includes an actuator arrangement including a forward component and a rear component, that the rear component is displaceable longitudinally relative to the housing, that the forward component is displaceable longitudinally relative to the housing and is angularly displaceable relative to the housing to an indexed, latching position in contact with the internal latching formation when first actuated, that the forward component is displaceable forwardly to a forward, third position in which it lifts the valve member off the seat and opens the valve to a rear position in the latching position by extending a member into an opening of the housing to contact and displace the rear component of the actuator arrangement.

According to yet another aspect of the present invention there is provided a medico-surgical tube including an inflatable sealing cuff encircling the tube, an inflation line in communication with the interior of the cuff and a valve according to any one of the preceding claims connected with the inflation line and arranged to be operable to allow or prevent gas flow to or from the cuff.

A tracheal tube including a valve according to the present invention will now be described, by way of example, with reference to the accompanying drawings, in which:

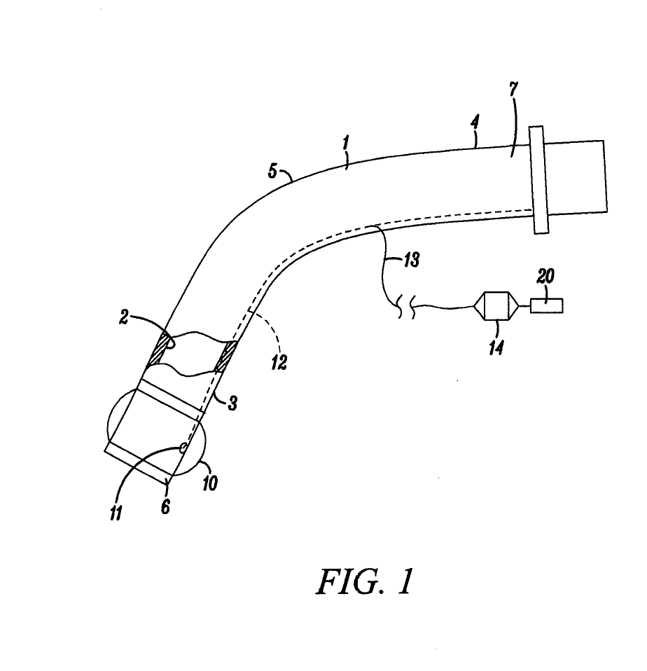

FIG. 1 is a side elevation view of a tracheostomy tube;

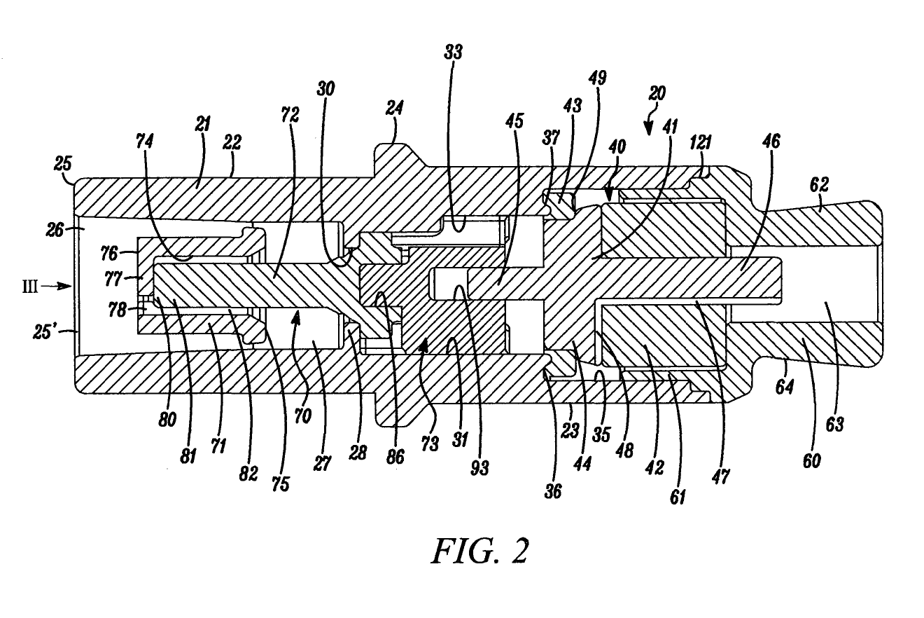

FIG. 2 is a cross-sectional side elevation view of the valve in an initial condition before actuation;

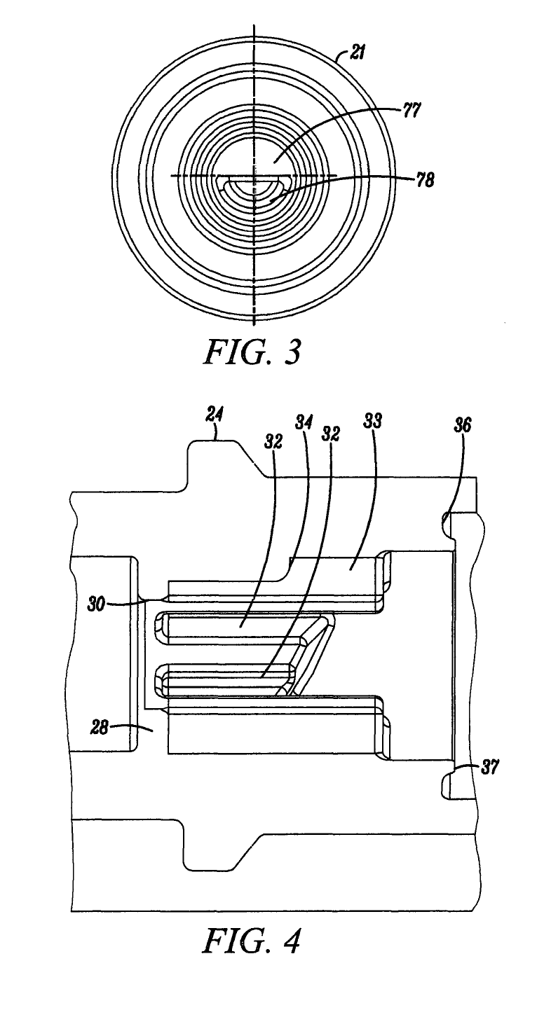

FIG. 3 is an end view of the valve along the arrow III of FIG. 2;

FIG. 4 is an enlarged side elevation view of a part of the inside surface of the valve housing;

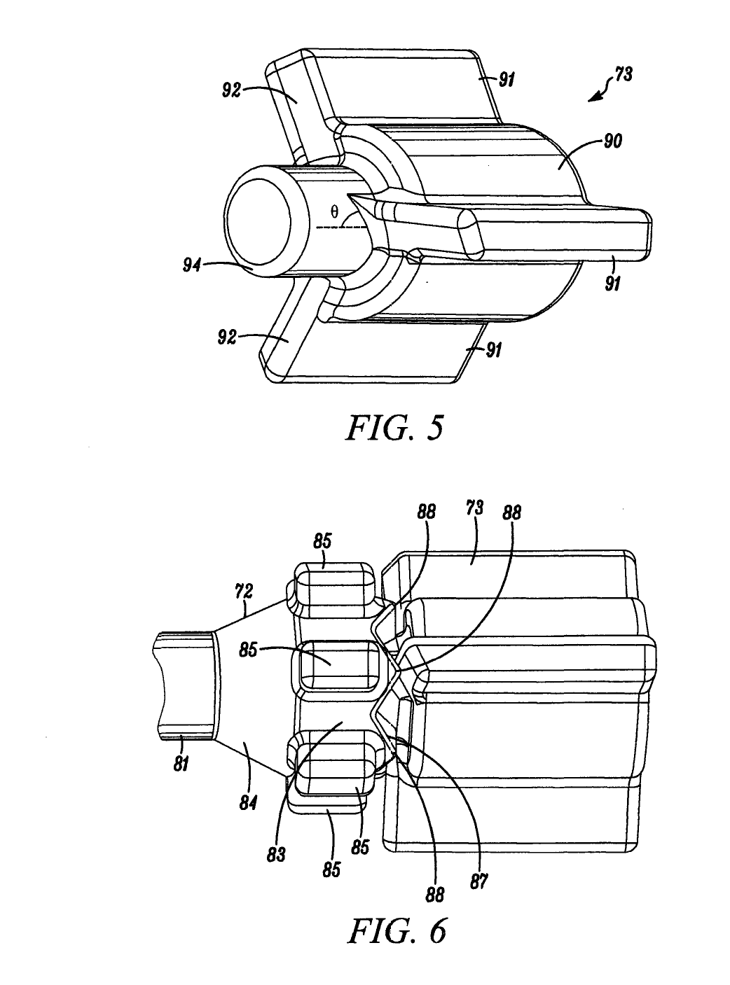

FIG. 5 is a perspective view of a forward actuator component inside the housing;

FIG. 6 is a side elevation view of the forward and rear actuator components within the housing; and

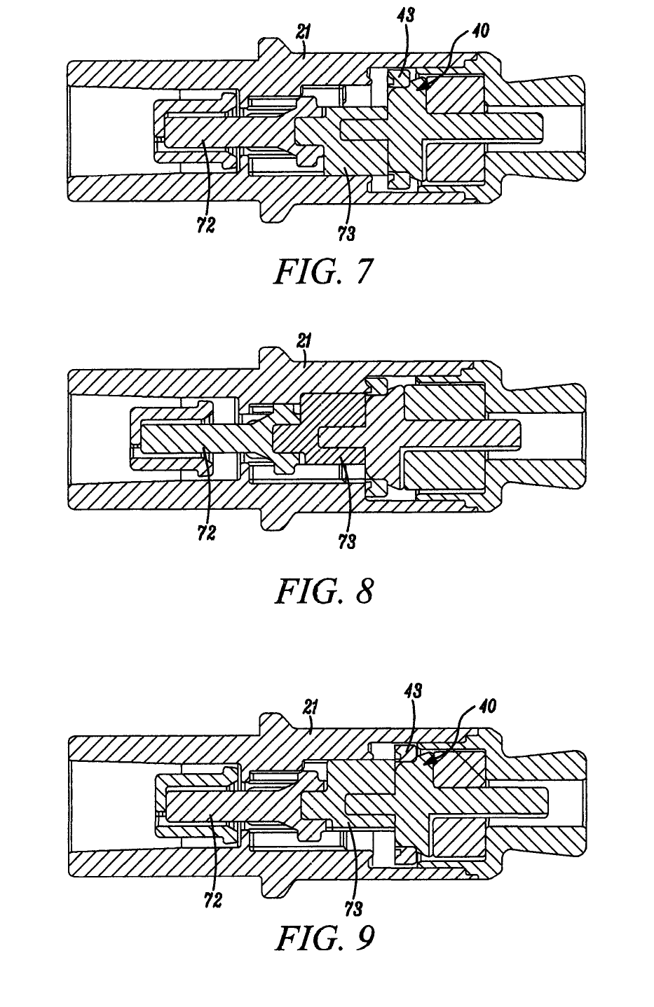

FIGS. 7, 8 and 9 are cross-sectional side elevation views of the valve in conditions subsequent to that shown in FIG. 2.

With reference first to FIG. 1, the tracheostomy tube includes a tubular shaft 1 having a bore 2 extending along its length. The tube is formed with a relatively straight patient end portion 3 and a relatively straight machine end portion 4 linked by a curved intermediate portion 5 so that the patient and machine ends 6 and 7 are angled at about 100° to one another. The shaft 1 is extruded or moulded from a plastics material such as PVC or silicone. Towards its patient end 6 the tube has sealing means provided by an inflatable cuff 10 embracing the shaft 1. The cuff 10 is of the high-volume/low-pressure kind so that it has a relatively floppy shape when deflated but, when inflated, it fills out at low pressure to a diameter just larger than the internal diameter of the trachea, so that it contacts the inside of the trachea with low pressure. The cuff 10 is moulded to the desired inflated shape from a plastics material such as PVC or polyurethane. The cuff 10 is attached to the shaft 1 at opposite ends over an opening 11 on the outer surface of the shaft into an inflation lumen 12 extending along the shaft within its wall thickness. The inflation lumen 12 is connected towards the rear end 7 of the tube with a small-bore inflation line 13 terminated by an inflation indicator 14 and a valve 20.

With reference now also to FIGS. 2 to 6, the valve 20 is of a generally cylindrical shape and circular section having an outer housing 21 of tubular shape with a rear portion 22, a forward portion 23 and a short, radially-projecting external annular flange 24 between them. The forward and rear portions 23 and 22 have a constant external diameter with the forward portion being slightly larger in diameter than the rear portion. The rear end 25 of the housing 21 opens into a luer-tapered bore 26, which extends along just over half the length of the rear portion 22 and continues as a section 27 of constant diameter to an internal ledge 28 located just to the rear of the external flange 24. The ledge 28 defines a circular opening 30 about half the diameter of the opening at the rear end 25. The bore through the forward part 23 of the housing 21, forwardly of the ledge 28, is divided into a rear section 31 and a forward section 35. The rear section 31 immediately adjacent the ledge 28 has a generally circular section and a diameter approximately equal to that of the section 27 on the rear side of the ledge. The section 31 adjacent the ledge 28, however, is interrupted by surface formations in the form of two sets of longitudinal grooves 32 and 33, shown most clearly in FIG. 4, and arranged alternately between one another around the housing. One set of grooves 32 extends to the forward face of the internal ledge 28; the other set of grooves 33 extends forwardly from a latching formation in the form of a ledge 34 located about midway along the central region along the housing. The two sets of grooves 32 and 33 are configured to serve a guiding, indexing and camming function the purpose of which will become apparent later.

The forward section 35 is larger in diameter and is divided from the rear section 31 by a forwardly-facing step 36 having a short, forwardly-extending wall 37 around its inner surface. This step 36 and wall 37 provide the seat of the valve.

The valve member 40 is formed of three parts, namely a core 41 of a hard plastics such as polyester, a drum-shape resilient member 42 of a non-metallic elastic material such as nitrile or neoprene and a sealing ring 43 of a soft, compressible material such as nitrile. The core 41 comprises a central, radially-extending disc 44, a first rearwardly-extending axial stem 45 and a second, forwardly-extending axial stem 46. The forward stem 46 has a groove 47 extending along its length that communicates with a radially-extending groove 48 formed in the forward face of the disc 44. The resilient member 42 is supported and retained on the forward stem 46 in abutment with the forward face of the disc 44. The grooves 47 and 48 allow a free flow of air between the inside of the resilient member 42 and the outside of the core 41. The sealing ring 43 is supported in a step 49 formed around the outer edge of the disc 44 with the rear face of the sealing ring engaging the valve seat provided by the step 36 and the wall 37. The valve member 40 is retained in the housing 21 by a forward end cap 60. The cap 60 has an externally-stepped sleeve 61 at its rear end extending inside and bonded to the forward end of the housing 21 in engagement with a step formation 121 around the forward end of the housing. The forward end of the cap 60 is in the form of a reduced diameter tubular extension 62 with a central bore 63 and an external tapered surface 64 that increases in diameter towards its forward end. The tubular extension 62 serves to receive one end of the cuff inflation line 13 extending from the inflation indicator 14.

The valve member 40 can be displaced forwardly in the housing 21 against the resilience of the resilient member 42 to unseat the sealing ring 43 from the valve seat 36 and 37 by means of an actuator assembly 70 so as to allow gas to flow between the sealing ring and the valve seat and along the grooves 47 and 48.

The actuator assembly 70 comprises three components, namely an adaptor 71, a rear cam component 72 and a forward cam component 73.

The adaptor 71 has a generally can shape with hollow interior 74, a circular section and an open forward end 75. The rear end 76 of the adaptor 71 is partially closed by a lateral floor 77 having a semi-circular opening 78 through it that opens into the interior 74. The adaptor 71 is secured on the rear end of the rear cam component 72 by an adhesive, solvent or heat bond between the inside face of the floor 77 and the rear end 80 of a cylindrical shaft 81 at the rear end of the rear cam component. The diameter of the shaft 81 is less than the internal diameter of the adaptor 71 so as to leave an annular gap 82 between the outside of the shaft and the inside of the adapter. This provides a gas path between the adaptor 71 and the rear cam component 72 via the opening 78, the annular gap 82 and the open forward end 75 of the adaptor. The forward end of the rear cam component 72 (shown most clearly in FIG. 6) is enlarged with a hollow cylindrical head 83 linked with the shaft 81 by a tapered portion 84. The annular outer surface of the head 83 has six, outwardly-projecting members in the form of short studs 85 of generally rectangular section spaced equally around its periphery. A short axial bore 86 extends along the head 83, opening at its forward end and closed at its rear end. The forward end face 87 of the head 83 is shaped with a saw-tooth configuration formed by six V-shape projections 88 the apices of which are aligned with the studs 85.

The forward cam component 73 is shown most clearly in FIG. 5. It has a main body 90 of cylindrical shape with three radially-projecting blades 91 equally spaced around its circumference. The blades 91 are generally rectangular, each having a rearwardly-facing end surface 92 inclined at an angle θ from the axis of about 60°. An axial bore 93 (FIG. 2) with a closed rear end extends along the major part of the main body 90 from its forward end. The length of the bore 93 is the same as that of the rear stem 45 of the core 41 of the valve member 40, the diameter of the bore being such as to receive the stem within it as a free sliding fit. The rear end of the forward cam component 73 has a short, axially-extending spindle 94 of circular section that is received in the bore 86 of the rear cam component 72 as a free sliding fit.

In the initial position, before use, as shown in FIG. 2, the valve member 40 is seated and closed, and the actuator assembly 70 is located at its rearmost position. In this position, the rear end of the studs 85 of the rear cam component 72 engage the forward side of the internal ledge 28 and the spindle 94 on the forward cam component 73 is extended fully within the bore 86 of the rear component. In this state, the six studs 85 of the rear component 72 and the three blades 91 of the forward component 73 lie in respective ones of the longitudinal grooves 32 formed around the interior of the housing 21. This prevents any relative angular displacement of either component 72 and 73 in the housing 21 but does not prevent axial displacement. It can be seen that, in this position, the forward end of the forward cam component 73 is spaced from the rear-facing surface of the disc 44 of the valve core 41 and that the rear end of the valve stem 45 is spaced by the same distance from the closed end of the bore 93 in the forward cam component.

When the valve is first actuated by inserting the nose of a syringe or other member into the rear end of the housing 21, this engages the adaptor 71 and pushes the actuator assembly 70 forwards. Displacement of the forward cam component 73 causes it to contact and engage the valve core 41 and then displace the valve member 40 forwardly and unseat the sealing ring 43 from the ledge 36 and wall 37. In this position fluid, such as air or other gas can flow along the valve, through the adaptor 71, around the outside of the actuator assembly 70, between the sealing ring 43 and the valve seat 36, 37, through the grooves 47 and 48 in the valve member 40 and out of the bore 63 in the cap 60, or in the opposite direction. During this forward displacement of the actuator assembly 70 there is no angular displacement of the components 72 and 73 of the assembly relative to one another or to the housing 21.

When the pressure exerted by the syringe or other member is reduced or removed, the resilience of the member 42 pushes the valve member 40 and the actuator assembly 70 rearwardly. In this forward position, the blades 91on the forward cam component 73 are unconstrained by the rear set of grooves 32 and are free to rotate relative to the rear cam component 72 in one direction. The V-shape formations 88 on the forward end 87 of the rear cam component 72 provide inclined surfaces that engage and cooperate with the inclined surfaces on the rear ends 92 of the blades 91 on the forward cam component 73 to urge it angularly in one direction relative to the rear cam component. This displaces the blades 91 out of alignment with the rear set of grooves 32 and into alignment with the latching formation or ledge 34 at the lower end of the other set of grooves 33. As the valve member 40 moves further to the rear, the forward cam component 73 comes to rest against the ledge 34, preventing further rearward movement, in the position shown in FIG. 8. In effect, the forward cam component 73 has been indexed or rotated through about 30° by the camming engagement between the component and the internal surface formations in the housing 21. In this second position the sealing ring 43 is again seated in a sealing fashion on the ledge 36 and wall 37, preventing any flow through the valve, that is, the valve is sealed or closed. The resilient member 42 applies a small pressure to hold the forward cam component 73 against the ledge 36, the sealing ring 43 not being in such a tight compression against the seat as in the initial position shown in FIG. 2. This is the normal sealed state or second position of the valve, the valve switching from the initial state shown in FIG. 2 to the second position shown in FIG. 8 following the first time the valve is actuated and opened.

When it is necessary to open the valve to add air to or remove air from the sealing cuff 10, the nose of a syringe (not shown) is inserted in the rear end 25 of the valve housing 21. The position of the rear end of the adaptor 71 along the bore 26, 27 is such that it is engaged by the forward end of the syringe nose and displaced to open the valve before the outer tapered surface of the syringe nose makes a sealing slip fit with the tapered surface 26 in the bore. When the nose of the syringe is sealed in the bore 26, the actuator arrangement 70 is in the third position with the valve member 40 open, as shown in FIG. 9 so that air can flow through the valve to inflate or deflate the sealing cuff 10. When the syringe is removed, the valve returns to the closed or sealed second position shown in FIG. 8 without any further rotation of the forward or rear cam components 73 and 72.

The arrangement of the present invention enables a non-metallic, MRI-compatible resilient element 42 to be used although the resilient element in the valve could be a conventional helical metal wire spring. The present invention provides an actuator in two parts 72 and 73 where one part 73 is latched into an operational position after an initial actuation. This arrangement allows for compensation of any dimensional change, especially shrinkage, of plastic components of the valve that might take place over time such as after sterilisation or caused by ageing after prolonged storage. The valve components can be readily manufactured using conventional techniques, such as injection blow moulding. The invention accommodates variations in tolerances between components without affecting the performance of the valve, thereby enabling manufacturing costs to be kept to a minimum.

1-10. (canceled)

11. A valve including an outer housing, a valve seat, a valve member displaceable along the housing into or out of sealing engagement with the valve seat, a resilient member arranged to urge the valve member into engagement with the seat, characterised in that the valve includes an actuator arrangement extending from the valve member to an opening of the housing, and that the actuator arrangement includes a first part displaceable from an initial position where the valve is closed to a second position where the valve is also closed and is subsequently displaceable from the second position to a third position where the valve member is displaced against the action of the resilient member away from the seat to open the valve and allow flow through the housing, the first part of the actuator arrangement returning to the second position when released.

12. A valve according to claim 11, characterised in that the actuator arrangement includes a second part displaceable axially relative to the first part, and that the valve is opened by pushing the second part axially relative to the housing.

13. A valve according to claim 12, characterised in that the forward end of the second part and the rear end of the first part have engaging inclined surfaces such that forward displacement of the second part can apply a rotational force to the first part.

14. A valve according to claim 12, characterised in that the second part has at least one outwardly-projecting member arranged to engage in a longitudinal groove along a part of the housing such as to enable longitudinal displacement of the second part but to prevent rotational movement.

15. A valve according to claim 11, characterised in that the first part has a plurality of outwardly-projecting members arranged to engage in respective longitudinal grooves along a part of the housing to prevent rotational movement when the valve is closed but to disengage the grooves when the actuator arrangement is engaged to open the valve and enable rotation of the first part to a position where the members engage on a latching formation when the actuator arrangement is released and thereby hold the first part in the second position.

16. A valve according to claim 11, characterised in that the resilient member is of a non-metallic material.

17. A valve according to claim 16, characterised in that the resilient member has a drum shape.

18. A valve including an outer housing, a valve seat, a valve member displaceable along the housing into or out of sealing engagement with the valve seat, a resilient member arranged to urge the valve member into engagement with the seat, characterised in that the outer housing has an internal latching formation, that the valve includes an actuator arrangement extending from the valve member to an opening of the housing such that the actuator arrangement can be displaced by extending a member into the opening of the housing, that the actuator arrangement includes a first part that is arranged to engage with the latching formation on the housing, that the first part of the actuator arrangement is displaceable from an initial position where the valve is closed to a second position where the valve is also closed and where the actuator arrangement engages with the latching formation to prevent subsequent displacement back to the initial position, that the first part of the actuator arrangement is displaceable from the second position away from the latching formation to a third position where the valve member is displaced against the action of the resilient member away from the seat to allow flow through the housing, and that the first part of the actuator arrangement is arranged to return to the second position in engagement with the latching formation when released.

19. A valve including an outer housing, a valve seat, a valve member displaceable along the housing into or out of sealing engagement with the valve seat, a resilient member arranged to urge the valve member into engagement with the seat, characterised in that the housing includes an internal latching formation, that the valve includes an actuator arrangement including a forward component and a rear component, that the rear component is displaceable longitudinally relative to the housing, that the forward component is displaceable longitudinally relative to the housing and is angularly displaceable relative to the housing to an indexed, latching position in contact with the internal latching formation when first actuated, that the forward component is displaceable forwardly to a forward, third position in which it lifts the valve member off the seat and opens the valve to a rear position in the latching position by extending a member into an opening of the housing to contact and displace the rear component of the actuator arrangement.

20. A medico-surgical tube including an inflatable sealing cuff encircling the tube, an inflation line in communication with the interior of the cuff and a valve connected with the inflation line and arranged to be operable to allow or prevent gas flow to or from the cuff, wherein the valve includes an outer housing, a valve seat, a valve member displaceable along the housing into or out of sealing engagement with the valve seat, a resilient member arranged to urge the valve member into engagement with the seat, an actuator arrangement extending from the valve member to an opening of the housing, the actuator arrangement including a first part displaceable from an initial position where the valve is closed to a second position where the valve is also closed and is subsequently displaceable from the second position to a third position where the valve member is displaced against the action of the resilient member away from the seat to open the valve and allow flow through the housing, the first part of the actuator arrangement returning to the second position when released.
